(19) United States
(12) Patent Application Publication
(10) Pub. No.: US 2009/0198333 A1
(43) Pub. Date: Aug. 6, 2009

(54) METHOD FOR TEXTURING THE SURFACE OF A SYNTHETIC IMPLANT

(76) Inventor: Hilton Becker, Boca Raton, FL (US)

Correspondence Address: LOWE HAUPTMAN HAM & BERNER, LLP, 1700 DIAGONAL ROAD, SUITE 300, ALEXANDRIA, VA, 22314, US

(21) Appl. No.: 12/169,000

(22) Filed: Jul. 8, 2008

Related U.S. Application Data
(63) Continuation-in-part of application No. 12/026,032, filed on Feb. 5, 2008.

(51) Int. Cl. A61F 2/12; A61B 19/00

(52) U.S. Cl. 623/8; 128/898

(57) ABSTRACT

A method for texturing the surface of a breast implant includes the step of partially impregnating a silicone outer surface of the implant with particles of a biologically active material such as acellular dermis of human or animal origin impregnated with hyaluronic acid. The biologically active material promotes tissue ingrowth into a plurality of cavities filled with a biologically active material.

### CROSS-REFERENCE TO RELATED APPLICATIONS

The present Application is a Continuation-In-Part of U.S. application Ser. No. 12/026,032, filed on Feb. 5, 2008, and priority is hereby claimed under 35 USC §120 based on these applications. Each of these applications are hereby incorporated by reference in their entirety into the present application.

### FIELD OF THE INVENTION

This invention relates to a method for texturing the surface of a synthetic implant using biologically active particles to form a textured surface that enhances tissue ingrowth and fixation to a breast implant for the reconstruction or augmentation of a human breast.

### BACKGROUND FOR THE INVENTION

Breast implants and methods for breast reconstruction and augmentation are well known and have been used for a period of over twenty years. In essence, the implants or prosthesis comprise a single or multi-lumen device with an envelope of medical grade elastomer such as silicone sized and adapted to receive a quantity of silicone gel, saline or the like. Some implants also include a tissue expansion feature such as an expandable bladder or lumen with a local or remote port to add or remove liquid or gel. Initially, soft pliable prosthetic implants included a smooth outer surface. However, it became apparent that the smooth surface prevented attachment of the capsule to the prosthesis.

One attempt to overcome such problems is disclosed in a U.S. Patent of Naficy (U.S. Pat. No. 4,298,998) for breast prosthesis with a biologically absorbable outer container. As disclosed, the prosthesis causes the capsule to form at a pre-determined controlled distance from the surface thereof. The prosthesis is constructed with a first phase or outer temporary component and a second phase or inner permanent component. The temporary outer component is a material which is absorbable under the conditions of use and an inert filler material preferably an absorbable biologically acceptable liquid such as saline filling the space between the inner and outer components. The outer portion is in the form of a sheet, film or coating of a material which can be absorbed in the body after surgical implantation.

A more recent development in breast implants is disclosed in the U.S. Pat. No. 5,964,803 of Ibersen et al. for an Enhanced Surface Implant and Method of Manufacture. As disclosed, an implantable body includes an outer membrane forming an enclosure for receiving a filler material. The surface of the outer membrane is characterized by a random distribution of peaks separated by valleys. The peaks and valleys are separated by gradual contour slopes with a smooth transition between the peaks and valleys which are substantially free from indentation. The enhanced surface provides improved anchoring of the implant yet reduces host reaction. The enhanced surface is formed by depositing a distribution of cured polymeric particles on an uncured tacky surface. This intermediate surface is then cured followed by application of a layer of uncured elastomeric material. The entire surface is then cured to form an enhanced surface.

A more recent patent of McGhan (U.S. Pat. No. 6,913,626) discloses a medical implant having a bio-absorbable textured surface. The McGhan patent discloses a hybrid medical implant having a bio-compatible non-absorbable core portion and a bio-absorbable textured outer surface portion overlying the core portion. The hybrid implant is useful as a prosthesis for breast augmentation and/or reconstruction. The core portion of the implant includes a body formed from a non-absorbable bio-compatible implantable material such as silicone or urethane elastomer.

The core portion may be a solid body, a viscous gel body or a fluid filled gel. The textured outer surface portion envelopes the core portion and presents an irregular bio absorbable textured surface to the exterior environment. As a capsule forms around the implant following implantation, the irregular contour of the outer surface of the implant disorients structural proteins in the capsule to impede suitable contraction thereof. Either during the formation of the capsule and/or after the capsule is formed, the outer bio absorbable surface portion of the implant is absorbed by the body of the host. After bio absorption of the bio-absorbable outer surface portion, the remaining core portion of the implant remains enveloped by the capsule but unattached to the capsular tissue. The outer bio-absorbable portion of the hybrid implant may include more than one bio-compatible bio absorbable material.

Notwithstanding the above it is Applicant's belief that there is a need and a potential market for an improved textured surface implant and a method for breast augmentation in accordance with the present invention. There should be a need and a market for such implants because they provide better anchoring and less problems with capsular contraction. Further, it is believed that such implants can be marketed at a competitive price, are durable, improve the rate of healing and lead to more satisfactory results.

### BRIEF SUMMARY OF THE INVENTION

In essence, the present invention contemplates a method for texturing the surface of a synthetic implant. The method includes the steps of providing a shaped mandrel and a dispersion of bio-compatible non-absorbable elastomer and repeatedly dipping the mandrel into the dispersion of bio-compatible non-absorbable elastomer and curing or partially curing the bio-compatible non-absorbable elastomer between sequential dips to thereby form an elastomer shell. The method also includes the steps of providing a mass of non-absorbable tissue growth enhancing particles such as acellular dermis of human or animal origin and forming an outer layer of bio-compatible non-absorbable elastomer on the elastomer shell. The non-absorbable tissue growth enhancing particles are then embedded in the outer layer with a portion of the particles forming irregular filled cavities (cavities in the outer layer filled with a tissue growth enhancing particles) and a second portion of the particles extending outwardly beyond a surface of the shell. The outer layer is then cured and when the implant is implanted into an individual, the partially embedded particles promote the growth of human tissue and blood vessels into the filled cavities, thus forming an interface of non-vascularized biological tissue and vascularized patient's tissue. While the tissue growth enhancing particles are referred to as non-absorbable it should be recognized that they may be partially absorbed. However, such particles are not rapidly absorbed as for example the salt used in the prior art preparation of a textured surface.

The present invention also contemplates a method for enhancing a human breast by augmentation that includes the step of providing an implant having a textured outer layer of silicone elastomer having a plurality of cavities filled with tissue growth enhancing material. Portions of the tissue growth enhancing materials protrude outwardly from the filled cavities. The implant also includes a hollow core filled with a fluid, gel or liquid of silicone, saline or soy and a layer or mass of a biologically active non-absorbable material such as non-absorbable acellular dermis. The method also includes the step of forming a capsular pouch from the mass of biologically active non-absorbable material, placing the implant into the pouch and implanting the pouch containing the implant behind the breast and reducing capsular contraction and rotation by a patient's tissue and blood vessels growing into the acellular dermis.

In a preferred embodiment of the invention, the acellular dermis, collagen combined with hyaluronic acid is partially impregnated in the outer layer of silicone elastomer and forms a mass of filled cavities or compartments so that the patient's blood vessels and tissue grow into the biologically active non-absorbable or only partially absorbable filled cavities to thereby anchor the implant in place. Polyglycans, collagen support material-naturally found in the dermis (deep layer of skin e.g. hyaluronic acid granules) are examples of materials that may be used as tissue growth enhancing particles individually or combined. The use of these materials on the surface of an implant produce a porous surface wherein the pores are filled with a biologically active material that promotes or enhances tissue growth into the pore with a biologically active material disposed therein.

The present invention also contemplates a method for enhancing a human breast or breasts by augmentation that includes the step of providing an implant having an outer layer of silicone elastomer and a hollow core filled with a fluid, gel or liquid of silicone, saline or soy and a layer or mass of a biologically active non-absorbable or partially non-absorbable tissue growth enhancing or promoting material such as non-absorbable or acellular dermis. The method also includes the step of forming a capsular pouch from the biologically active non-absorbable material, placing the implant into the pouch and implanting the pouch containing the implant behind the breast to thereby reduce capsular contraction and rotation. The reduction in capsular contraction and rotation is due to a patient's tissue and blood vessels growing into the biologically active material. In a preferred embodiment of the invention, the biologically active particles are partially impregnated in the outer layer of silicone elastomer and form a mass of acellular dermis filled cavities or pores. The patient's blood vessels and tissue grow into the biologically active non-absorbable filled cavities to thereby anchor the implant in place.

Further, the invention contemplates an implanted breast prosthesis for breast reconstruction and augmentation. The prosthesis includes a closed envelope of the type including an outer layer of silicone elastomer and a hollow core surrounded by the outer layer. The implants are then filled with silicone, silicone gel, saline or soy in a conventional manner. The implant also includes a meshed dermal cover of a biologically active non absorbable material that covers at least a forward portion of the implant. In a preferred form, the meshed dermal cover is a mass of acellular dermis particles wherein a portion of the particles are partially impregnated or imbedded in the layer of silicone elastomer to thereby form a large number of particle filled cavities formed in the layer of silicone elastomer.

The invention will now be described in connection with the following drawings wherein like numbers have been used to identify like parts.

### DESCRIPTION OF THE DRAWINGS

FIG. 1 is a flow chart illustrating a method for forming a textured implant in accordance with the present invention.

FIG. 2 is a schematic illustration of the use of an acellular dermal sheet used as a layer between body tissue and the implant;

FIG. 3 is a schematic illustration of a breast prosthesis as used in one embodiment of the present invention wherein an acellular dermal sheet covers the whole implant;

FIG. 4 is a schematic illustration of a breast prosthesis as used in a second method for breast augmentation wherein an acellular meshed bag cover the whole implant and used to suspend the implant;

FIG. 5 is schematic illustration of a further embodiment of the invention wherein a meshed dermal sheet is adhered to the implant;

FIG. 6 is a perspective view of a breast prosthesis in accordance with a first embodiment of the invention;

FIG. 7A is a schematic illustration of a silicone sheet impregnated with dermal particles;

FIG. 7B is a schematic illustration of a silicone sheet with dermal particles mixed with silicone as a paste and applied to the silicone sheet and dried;

FIG. 7C is a magnified view of the silicone sheet with dermal particles as shown in FIG. 7B;

FIG. 8A is a schematic illustration of a conventional breast implant;

FIG. 8B is a schematic illustration of a conventional breast implant including a dermal sheet implanted in a human breast;

FIG. 8C is a schematic illustration of a breast implant including a meshed dermal sheet and implanted in a human breast;

FIG. 8D is a schematic illustration of a breast implant wrapped with a dermal sheet and implanted in a human breast;

FIG. 8E is a schematic illustration of a breast implant wrapped in a meshed dermal sheet and implanted in a human breast;

FIG. 8F is a schematic illustration of a breast implant impregnated with dermal tissue and implanted in a human breast;

FIG. 9A is a schematic illustration of an implant with a smooth surface covered with a muscle;

FIG. 9B is a schematic illustration showing a human breast implant covered with a dermal sheet;

FIG. 9C is a schematic illustration of an implant including absorbable particles;

FIG. 9D is a schematic illustration showing a human breast implant surface with a plurality of cavities after absorption of the particles shown in FIG. 9C and with limited tissue growth into the cavities;

FIG. 9E is a schematic illustration of an implant surface impregnated with biologically active dermal particles embedded therein;

FIG. 9F is a schematic illustration showing the ingrowth of tissue and blood vessels into the cavities filled with a biologically active material in accordance with the present invention; and

FIG. 10A is a schematic illustration of a silicone base having a particle of sponge form collagen impregnated therein;

FIG. 10B is a schematic illustration of a sponge form collagen impregnated in a silicone base or layer of an implant wherein hyaluronic acid has been added to the cavities in the sponge form collagen; and

FIG. 10C is a schematic illustration showing the sponge form collagen of FIG. 10B with cells migrating into the hyaluronic acid filled cavities and blood vessels growing into the filled cavities.

### DESCRIPTION OF THE PREFERRED EMBODIMENTS OF THE INVENTION

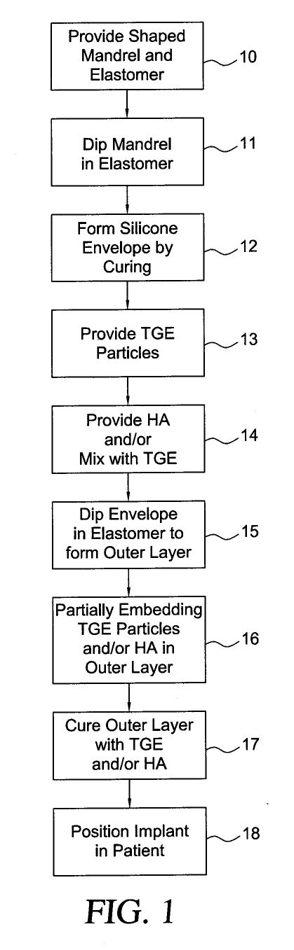

A method for texturing an outer layer or surface of a synthetic implant such as a silicone breast implant will now be described with reference to FIG. 1. The method includes a first step 10 of providing a shaped mandrel and a dispersion of bio-compatible non-absorbable elastomer such as silicone. In a second step 11 the shaped mandrel is repeatedly dipped into the dispersion to coat the mandrel with the bio-compatible non-absorbable elastomer. These first two steps are the same as used in producing conventional implants.

In step 12, a silicone envelope or shell is formed by curing the elastomer as for example between successive coatings or dips. In steps 13 and 14 a mass of tissue growth enhancing particles and/or hyaluronic acid granules are provided. In step 15 the envelope from step 12 is dipped into elastomer to form an outer layer and before curing, the particles from steps 13 and 14 are partially embedded in an outer layer in step 16 and form a plurality of irregular shaped cavities therein with the tissue growth enhancing particles and/or hyaluronic acid granules fill the cavities and the layer is cured in step 17. Then, in step 18 the implant, including an outer layer with partially embedded particles, is implanted in a patient in step 18.

The invention also contemplates a method for promoting ingrowth of human tissue and blood vessels into a silicone breast implant. The method includes the step of forming a silicone breast implant having an outer portion or layer creating a plurality of irregular shaped pores therein with a non-absorbable tissue growth promoting material as for example acellular dermis of human or animal origin. The materials may include collagen, cross-linked collagen, collagen precursors, collagen support material and hyaluronic acid granules.

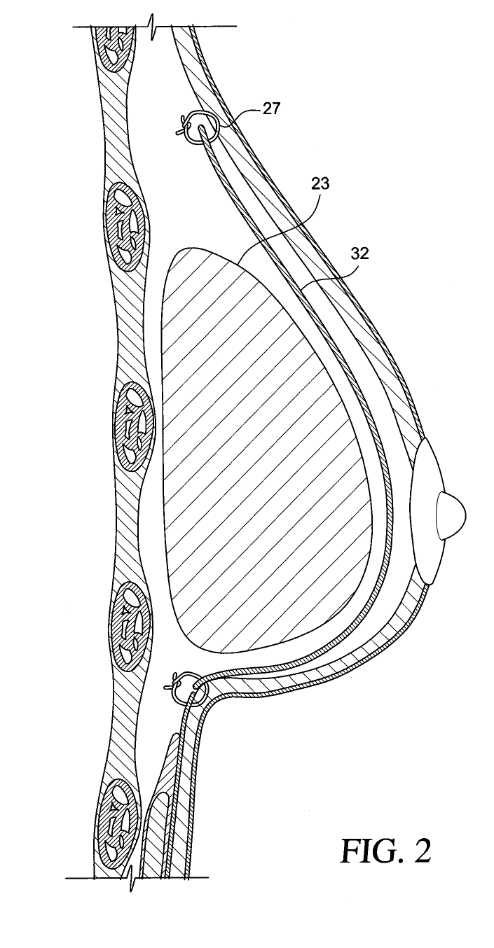

FIG. 2 illustrates a conventional technique that utilizes a collagen sheet 32 sutured to an upper portion of a patient's breast and extends downwardly and forwardly of the implant 23. The collagen sheet 32 is stitched to an upper portion of the breast as indicated by 27.

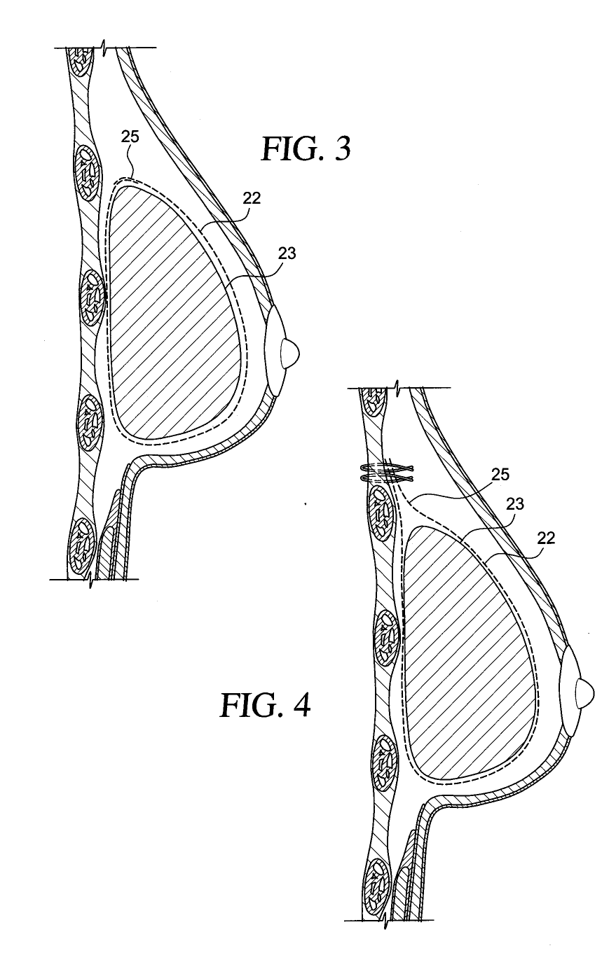

As shown in FIG. 3 an improved breast implant 20 in accordance with one embodiment of the present invention includes a meshed dermal pouch 22 of a cross-linked collagen with an inner non-absorbable prosthesis enclosed or encased therein. This represents the second stage in the evolution of the process. As shown, the implant includes a tube 24 leading to a pouch (not shown) but held within the surgeon's hand 26. The remote port and tube 24 are of conventional design and typically used to insert additional saline or silicone fluid or reducing the fluid filler from the inner prosthesis. The inner non-absorbable prosthesis or implant 23 is shown in FIGS. 3 and 4 wherein the inner prosthesis or inner implant 23 is encased in the dermal pouch 22. As shown in FIG. 3, the dermal pouch is closed in an upper portion 25 to provide a fully enclosed structure. In FIG. 4 the pouch 22 includes an upper portion extending up above the inner pouch 23 and upper portion 25 which is sutured to a chest muscle or the like to position the dermal pouch 22 within a human breast.

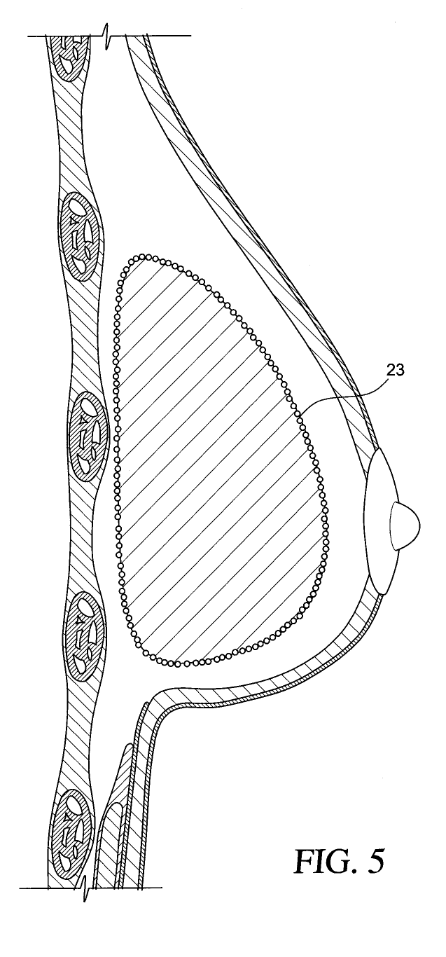

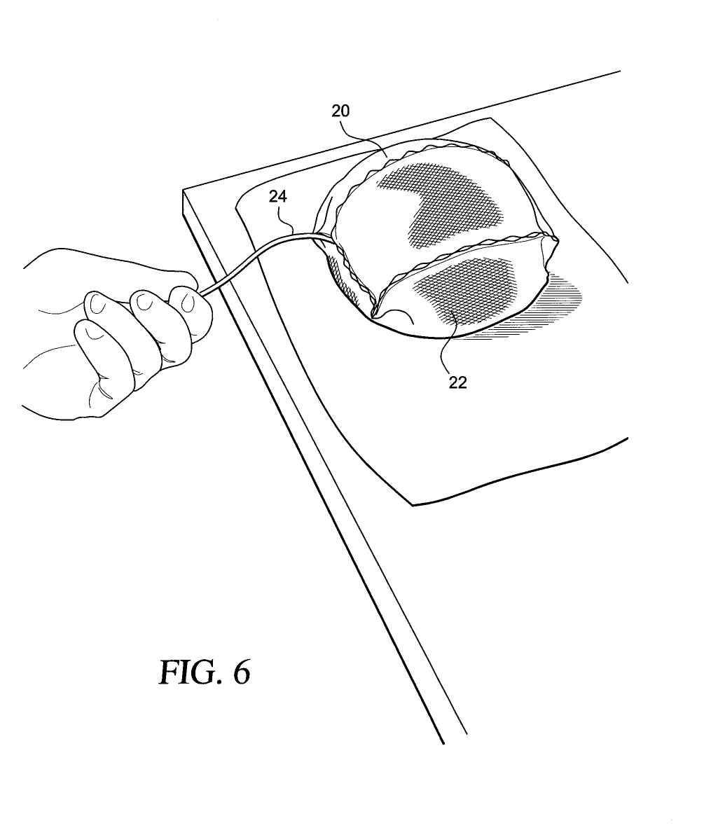

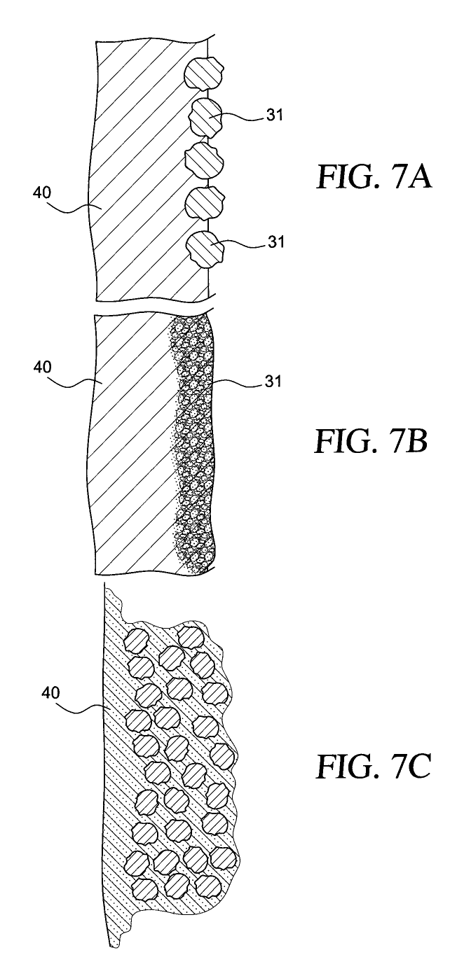

FIGS. 5 and 6 illustrate two embodiments of the invention. For example, in FIG. 5 the inner non-absorbable prosthesis or implant is coated with a plurality of particles that are partially impregnated into the outer surface of the implant 23. The coating with dermal particles impregnated onto a silicone shell as shown in FIG. 5 is illustrated more clearly in FIG. 7A-7C. For example, the dermal particles 31 may be mixed as a paste with liquid silicone and then applied to a silicone sheet and dried. As a result, the silicone sheet is partially impregnated with a plurality of dermal particles and forms a plurality of cavities in the silicone sheet. As shown in FIG. 7C the dermal particles 31 are disposed on the silicone sheet or layer 40 and attached to a patient's tissue.

FIG. 6 illustrates the use of a pouch made of a collagen sheet and fully enclosing the implant. The implant 20 is contained in a pouch 22 of dermal material. As illustrated a filling tube 24 for expanding or contracting the size of the implant leads to a surgeons hand.

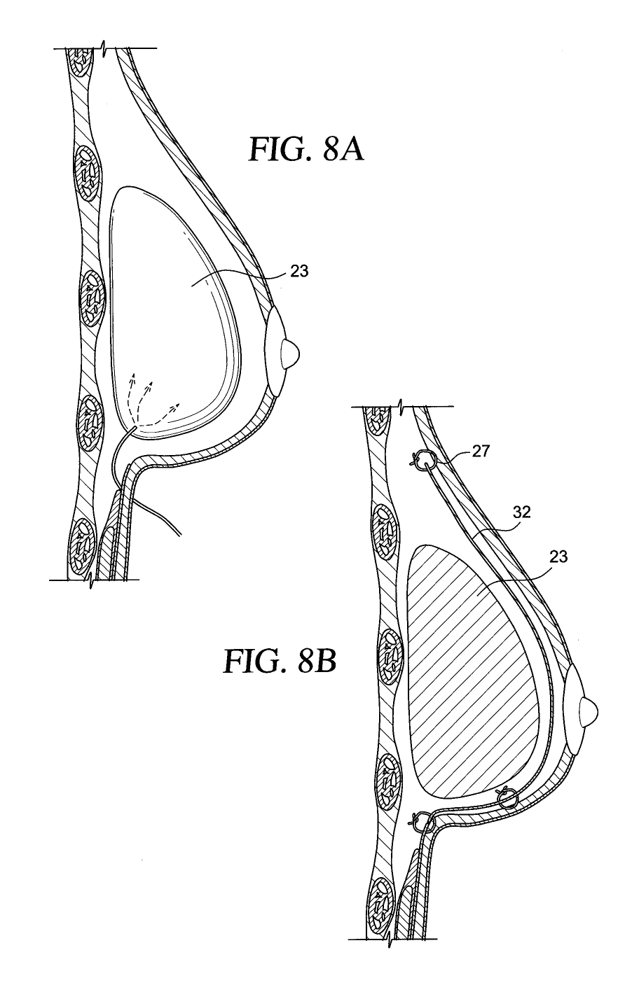

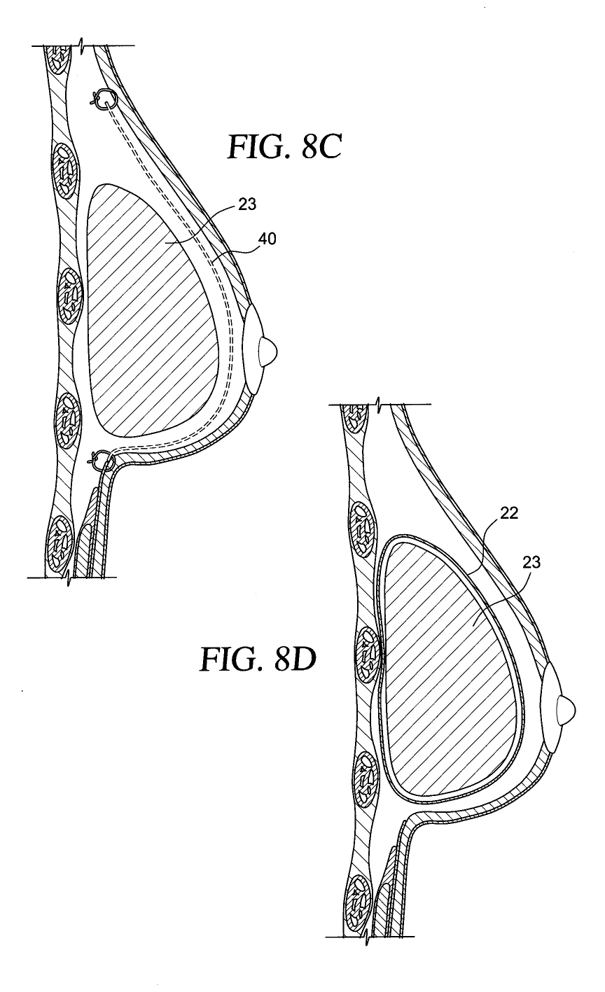

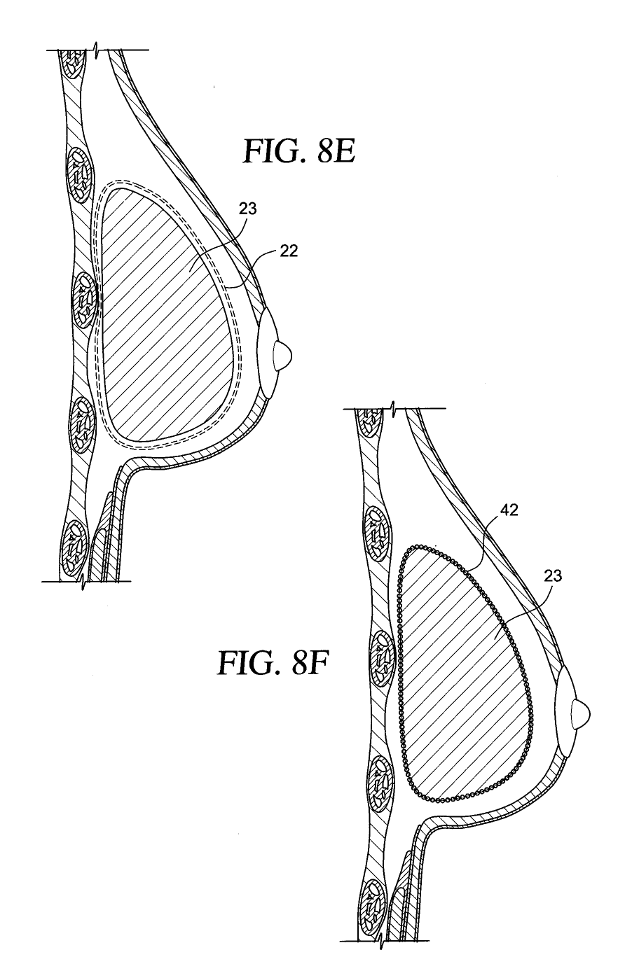

The evolution of various embodiments of the present invention is illustrated in FIGS. 8A-8F wherein FIG. 8A shows a smooth sided implant disposed in a human breast. By comparison the same implant includes a dermal sheet of collagen or the like disposed in a forward portion of the breast in the same manner as shown in FIG. 2. In FIG. 8C the dermal sheet of collagen or the like 32 in FIG. 8B is replaced with a meshed dermal sheet 40. Further, FIG. 8D illustrates a smooth sided internal implant encased in a dermal pouch 22 while FIG. 8E shows the same inner implant disposed in a meshed dermal pouch 40. Finally, FIG. 8F illustrates an implant coated with a dermal paste 42 that is partially implanted into the outer layer of the implant.

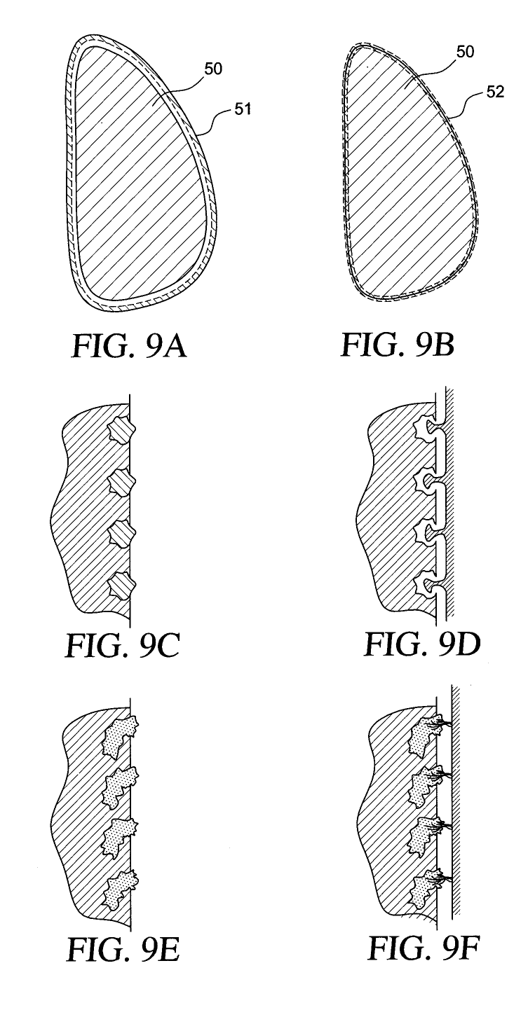

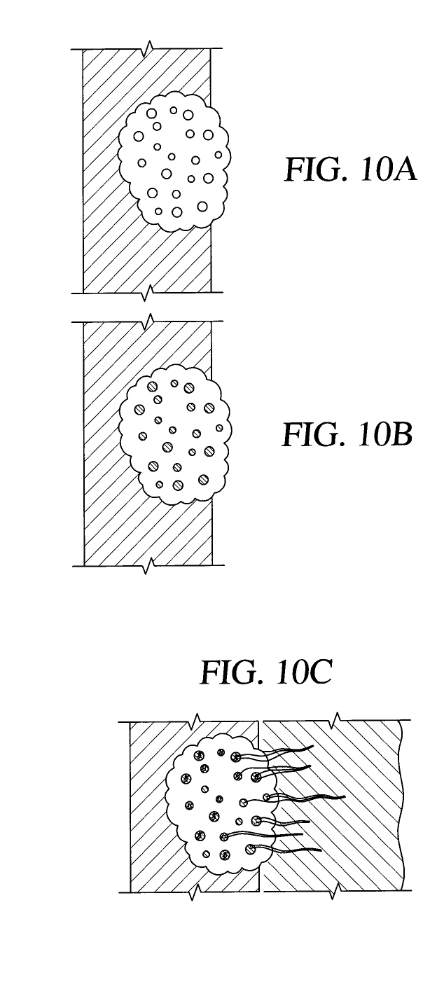

FIGS. 9A-9F are schematic illustrations of two forms of implants and the surfaces of the two forms of implant before and after implantation. For example, FIG. 9A shows an implant 50 having an outer layer of absorbable particles 51 while FIG. 9B shows an implant 50 with a plurality of non-absorbable tissue growth enhancing particles 52 embedded in the outer surface thereof. FIG. 9C shows the absorbable particles 51 partially embedded in the surface of the implant 50 before being implanted into a patient. For comparison, FIG. 9D shows limited ingrowth of a patient's tissue and/or blood vessels 60 into the cavities 61 formed when the particles 51 are absorbed by the patient's body. FIG. 9E illustrates the outer surface of the layer 62 impregnated with non-absorbable tissue growth enhancing (TGE) particles 65 that are generally embedded in the surface with portions thereof extending outwardly from the surface thereof. For contrast FIG. 9F illustrates the more extensive ingrowth of a patient's tissue and blood cells into the chambers formed and filled by the particle 65.

The devices in accordance with the present invention are partially impregnated into the outer layer of an implant and promote ingrowth of a body's tissue and blood vessels into the biologically active material between the implant bodies and the tissue to form a biological barrier between the patient's tissue and the silicone implant. Furthermore, a relatively strong bond between the patient's tissue and the implant may form. This reduces the likelihood of implant rotation and capsular contraction.

The plurality of particles are preferably applied to the final or outer coating before curing thereof. A portion of the particles then project outwardly from the shell and provide an irregular topography having a plurality of irregular shaped cavities that are filled by portions of the particles.

The method for texturing the surface of a silicone breast implant that enhances tissue ingrowth involves the use of acellular dermis derived from cadaver sources, e.g. AlloDerm® or fetal calf sources, e.g. SurgiMend™ to provide additional coverage for implants in reconstructive breast surgery.

The acellular dermis consists essentially of strands of collagen into which a patient's cells and blood vessels migrate. The graft is essentially incorporated into the patient's tissue. The process of vascularization may be accelerated by making cavities or holes in the dermis material. Further, by adding a suitable growth enhancing matrix such as hyaluronic acid to the cavities the process of tissue ingrowth can be further facilitated.

Initally, acellular dermal sheets were placed over the implant. The next step was to use meshed dermis i.e. a dermal sheet with a plurality of holes therein. The implants were then placed in a meshed bag or pouch. In a preferred embodiment of the invention, a hyaluronic acid matrix was placed in the holes and the meshed material containing hyaluronic acid was adhered to the implant as a sheet or in a preferred form by implanting tiny particles into a silicone layer.

An important point is that the particles are left embedded into the silicone rather than being dissolved out to result in a textured surface to decrease capsular contraction. Tissue then grows into the empty spaces after the bio-absorbable material is absorbed. The collagen (acellular dermis) particles are not absorbed but rather become incorporated into the host tissue.

The collagen particles may be used alone or preferably with hyaluronic acid. The hyaluronic acid may also be cross-linked to prevent absorption and in this form used alone.

It has also been recognized that hyaluronic acid granules provides a favorable environment for cell growth and motility that fosters tissue regeneration. Other ground substances such as polyglycans and materials from connective tissue or hydroxylappetite may be used, but collagen and/or hyaluronic acid granules are preferred. It has also been recognized that hyaluronic acid has therapeutic implications in that it may modulate the healing of adult wounds in a manner more similar to the healing of fetal wounds. See “Using Hyaluronic acid granules to Create a Fetal-like Environment Envitreo” by Scott Shepherd MD, Hilton Becker MD and James Hartman PhD, Annals of Plastic Surgery, 1996, Volume 36, lines 65 to 69. As stated therein, “Highly Purified Medical Grade H.A. extracted from the cell walls of select streptococcus strains is currently employed to reduce the incidence of post-operative adhesions, viscolelastic agent in intraocular surgery, a synovial replacement device and in various cosmetic applications.”

It is well known that texturing of silicone implants prevents rotation of anatomical or shaped implants and that textured surfaces also decrease capsular contracture (hardening around the implant). A number of methods exist for creating a textured surface. For example, placing salt on the surface of the silicone shell before drying and then dissolving the salt in water has been used. The cavities left by the salt create a textured surface. Various types of imprints can also be mechanically stamped on the surfaces of a shell before drying. In the present invention the above methods of texturing may be used to form a textured surface. However, in a preferred embodiment of the present invention, collagen particles and/or hyaluronic acid granules are partially embedded in an outer silicone layer. The particles not only create a textured surface, but the silicone is partially concealed or protected from tissue ingrowth. Further, it has been shown that collagen will adhere to the surrounding tissues and that covering tissue and blood vessels grow into the collagen to thereby protect the patient from adhesions while preventing rotation and hardening.

A still further approach for providing a tissue growth enhancing implant contemplates forming a textured surface as for example by a prior art method, dissolving the salt and then immersing either at the time of manufacture or shortly before implantation the textured shell is immersed in a hyaluronic gel.

A further embodiment of the invention contemplates the addition of an antibiotic and/or steroid to the shell of the prosthesis or impregnated into the silicon layer together with hyaluronic acid and/or with the collagen particles to further promote healing and/or prevent infection.

While the invention has been described in connection with its preferred embodiments, it should be recognized that changes and modifications may be made therein without departing from the scope of the appended claims.

What is claimed is:

1. A method for texturing the surface of a synthetic implant comprising the steps of:
providing a shaped mandrel and a dispersion of bio-compatible non-absorbable elsatomer;
repeatedly dipping the mandrel into the dispersion of bio-compatible non-absorbable elastomer and curing the bio-compatible non-absorbable elastomer to thereby form an elastomer shell;
providing a mass of non-absorbable or partially non-absorbable tissue growth enhancing particles and forming an outer layer of bio-compatible non-absorbable elastomer on said elastomer shell;
partially embedding the non-absorbable or partially non-absorbable tissue growth enhancing particles in said outer layer with a portion of said particles forming irregular filled chambers within said outer layer and a second portion of the particles extending outwardly beyond a surface of the shell; and
curing the outer layer whereby the partially embedded particles promote the growth of human tissue into the chambers when implanted in a human body.

2. A method for texturing the surface of a synthetic implant according to claim 1 wherein said implant is a breast implant for breast augmentation and wherein said dispersion of bio-compatible non-absorbable elastomer is silicone.

3. A method for texturing the surface of a synthetic implant according to claim 2 in which the mass of non-absorbable tissue growth enhancing particles are selected from the group consisting of collagen, cross-linked collagen, collagen precursors, collagen support material, hyaluronic acid and cross-linked hyaluronic acid.

4. A method for texturing the surface of a synthetic implant according to claim 3 in which said mass of non-absorbable tissue growth enhancing particles include hyaluronic acid granules for forming a gel when implanted in the human body.

5. A method for texturing the surface of a synthetic implant according to claim 4 in which said mass of non-absorbable tissue growth enhancing particles includes collagen.

6. A method for texturing the surface of a synthetic implant according to claim 5 in which said mass of non-absorbable or partially non-absorbable tissue growth enhancing particles include sponge form collagen having a plurality of holes therein and wherein said holes are filled with or partially filled with hyaluronic acid.

7. A implantable breast prosthesis with a textured surface for breast augmentation, said prosthesis comprising:
a silicone elastomer shell including an outer layer of silicone elastomer, a hollow core and silicone or saline fluid dispersed within said hollow core; and
said outer layer including a plurality of irregularly shaped non-absorbable tissue growth enhancing particles partially embedded in said outer layer with portions of said particles forming an irregular shaped chamber in said outer layer with said chamber filled by said particles and a second portion of said particles extending outwardly from said outer layer so that tissue growth is enhanced when said implant is implanted in a human body.

8. A implantable breast prosthesis according to claim 7 wherein said plurality of non-absorbable tissue growth enhancing particles are selected from the group consisting of collagen, collagen precursors, collagen support material, hyaluronic acid granules and mixtures thereof.

9. A implantable breast prosthesis with a textured surface for breast augmentation according to claim 8 in which said non-absorbable tissue growth enhancing particles include hyaluronic acid granules for forming a gel when implanted in the human body.

10. A implantable breast prosthesis with a textured surface for breast augmentation according to claim 8 in which said non-absorbable tissue growth enhancing particles include hyaluronic acid and collagen.

11. A method for augmentation of a human breast comprising the steps of providing a breast implant having a hollow core filled with silicone or saline and an outer layer of silicone elastomer surrounding said hollow core, and a mass of biologically active non-absorbable or partially non-absorbable tissue growth enhancing particles;
forming a plurality of hollowed out spaces filled with portions of the biologically active non-absorbable or partially non-absorbable tissue growth enhancing particles with portions of the biologically active non-absorbable or partially non-absorbable tissue growth enhancing particles extending outwardly from the outer layer to thereby form a breast implant with a textured tissue growth enhancing surface;
inserting the implant with a textured tissue growth enhancing surface behind a patients breast to thereby promote the patient's tissue and blood vessels to grow into the hollow spaces filled with biologically active non-absorbable or partially non-absorbable tissue growth enhancing particles.

12. An implantable prosthesis for breast augmentation, said prosthesis comprising:
a closed envelope including an outer layer of silicone elastomer, a hollow core and a silicone or saline fluid dispersed within said hollow core;
a dermal cover of a biologically active absorbable or partially non-absorbable tissue growth promoting material covering a portion of said envelope so that a patient's tissue and blood vessels grow into said cover for reducing capsular contraction and rotation of the implant.

13. An implantable prosthesis for breast augmentation according to claim 12 in which said cover is a mesh cover selected from the group consisting of collagen, collagen precursors, collagen support material, hyaluronic acid granules and mixtures thereof.

14. An implantable prosthesis for breast augmentation according to claim 13 in which said cover is in the form of a pouch and in which said closed envelope is placed within said pouch.

15. An implantable prosthesis for breast augmentation according to claim 13 in which said mesh cover is partially embedded in said outer layer of silicone elastomer.

16. An implantable prosthesis according to claim 13 in which said mesh cover is made of collagen.

17. An implantable prosthesis according to claim 13 in which said cover includes hyaluronic acid granules crystals.

18. An implantable prosthesis according to claim 13 in which said cover material include a mixture of sponge form collagen and hyaluronic acid.

19. A method for promoting the ingrowth of human tissue and blood vessels into a silicone breast implant comprising the steps of:
forming a silicone breast implant having an outer layer of silicone and a plurality of irregular shaped pores in the outer layer;
filling the pores in the outer layer with a non-absorbable or partially non-absorbable tissue growth promoting material; and
implanting the implant into a human breast.

20. A method for promoting the growth of human tissue and blood vessels into a silicone breast implant according to claim 19 which includes the step of adding an antibiotic and/or steroid to said outer layer of silicone and/or into said irregular shaped pores in said outer layer.
